(19) United States
(12) Patent Application Publication
(10) Pub. No.: US 2011/0190639 A1
(43) Pub. Date: Aug. 4, 2011

(54) Bi-Spectral Peroperative Optical Probe

(75) Inventors: Philippe PELTIE, Saint Paul de Varces (FR); Michel BERGER, Claix (FR)

(73) Assignee: Commissariat A L'Energie Atomique et aux Energies Altematives, Paris (FR)

(21) Appl. No.: 13/019,119

(22) Filed: Feb. 1, 2011

(30) Foreign Application Priority Data
Feb. 2, 2010 (FR) 10 00401

(51) Int. Cl. A61B 6/00

(52) U.S. Cl. 600/476

(57) ABSTRACT

Optical probes for medical applications are provided. The probe is devised so as to be able to be held in one hand. A basic version of the probe includes: a first excitation lighting source suitable for causing a fluorescence radiation of predetermined substances; a second visible lighting source, the first and the second source being devised so as to illuminate a common zone termed the intervention zone; a first photosensitive matrix sensor; and a second photosensitive matrix sensor. The first and second photosensitive matrix sensors are devised in such a way that, when the optical probe is arranged a predetermined distance from the intervention zone, the image in the visible spectrum of the said zone is formed on the photosensitive surface of the first matrix sensor and the image in the fluorescence spectrum of the said zone is formed on the photosensitive surface of the second sensor. A first variant of the probe includes only a single optical objective, a second variant only a single photosensitive matrix sensor, and a third variant makes it possible to work under polarized light.

### CROSS-REFERENCE TO RELATED APPLICATION

This application claims priority to foreign French patent application No. FR 1000401, filed on Feb. 2, 2010, the disclosure of which is incorporated by reference in its entirety.

### FIELD OF THE INVENTION

The field of the invention is that of peroperative optical probes used in immunophotodetection techniques. They serve during surgical intervention and notably for the ablation of tumours.

### BACKGROUND

The technique of immunophotodetection (acronym: IPD) was initiated about ten years ago, notably at the Centre de Recherche et de Lutte contre le Cancer in Montpellier. The principle of this technique consists in injecting into a live body, human being or animal, an antibody-fluorophore conjugate which fixes to cancerous cells through an antibody-antigen reaction. For example, digestive cancers may secrete so-called carcinoembryonic antigens (acronym: CEA) which serve as targets for the antibodies. In the course of the operation, the surgeon must therefore be able to detect the fluorophores indicating the presence of diseased cells. Basically, this detection is ensured by a probe which comprises a fluorophores excitation laser source and a photo-detector whose spectral sensitivity range is adapted to the fluorescence spectrum of the said fluorophores. This technique is also known by the term “Fluorescence Reflectance Imaging” or “FRI”.

This technique presents several difficulties that have to be surmounted in order to present a quality image to the surgeon allowing him to make the most effective possible moves. A first difficulty is related to the autofluorescence of living tissues. It is known that the autofluorescence of tissues is significant when they are illuminated by radiation whose spectrum lies in the visible. Such is typically the case for operating theatres which are illuminated either by natural light, or by the light from lighting sources such as “neon” lamps or “halogen” lamps. Hence, if no particular precautions are taken, the environmental light may cause serious nuisance to the useful, but always very weak, fluorescence signal. A simple calculation makes it possible to understand this difficulty. In a peroperative probe, the mean power density given by the excitation source at 700 nm does not exceed 25 μW/mm². If the fluorophore has a quantum yield of 0.1, a concentration of 10 nM and if, on the other hand, the tissue thickness traversed does not exceed 0.1 mm, then the fluorescence intensity equals approximately 0.26 10⁻⁴ of the power density, i.e. about 0.6 10⁻⁶ mW/mm². Now, operating theatre lighting of scialytic type gives a power density of 0.4 mW/mm², a million times more powerful. To solve this problem, a first solution consists in using powerful excitation laser sources to improve the fluorescence. In this case, it is necessary to ensure the ocular safety of the people present during the intervention. A second possible solution is to use filtered light that is highly adapted to the specifics of this “Fluorescence Reflectance Imaging” technique. The devices described in the publication referenced US2005/0182321 entitled “Medical imaging systems” comprise arrangements of this type. Indeed, the system described and represented in FIG. 1 of this publication comprises two filtered lighting sources, the first ensuring visible lighting, the second ensuring excitation lighting intended for tissue fluorescence. This system also comprises two cameras having a common optical axis, the first dedicated to fluorescence radiation lying in the near infrared, the second dedicated to visible radiation.

A second problem is related to the surface to be examined which may be, for example, an abdominal cavity. Generally, the latter is vast and if a fixed probe is used, then an image of the whole of the abdominal cavity must be produced. In this case, the resolution given by the camera is poor and there is a risk of the surgeon not seeing cancerous nodules if they are of overly small dimensions or if they are hidden, the nodules of significant size having been detected either by eye, or by palpation. Hence, it is preferable to use a portable probe that the surgeon will be able to move over the surface to be examined, the objective being to detect nodules whose size does not exceed a few tenths of a millimetre. The publication WO 02/061405 entitled “Method and hand-held device for fluorescence detection” describes such a probe. However, the fluorescence detection carried out by this probe is rudimentary. It is ensured by a simple photo-detector which does not make it possible to produce a genuine image of the zone to be analysed and which simply gives an indication of the presence or otherwise of fluorescent zones. Moreover, the problem of nuisance autofluorescence is not solved in such a probe.

A third problem is that of the robustness of the probe. Such a probe being intended for intense use, the optical adjustments should be as robust as possible.

Finally, a fourth problem is the quality of the visible image of the biological tissues illuminated by point sources. Indeed, it has been noted that, on account of the moistness of these tissues, they behave like reflecting surfaces, thus giving rise to appreciable specular reflection. This reflection may considerably degrade the visible image.

### SUMMARY OF THE INVENTION

The optical probe according to the invention makes it possible to alleviate these various drawbacks. It is, indeed, portable, unites in a single compact module at one and the same time the excitation and lighting sources and the two cameras dedicated on the one hand to visible imaging and on the other hand to fluorescence imaging and finally comprises means making it possible to effectively filter the specular reflections. It also comprises devices ensuring ocular safety. Moreover, it is possible to fix a viewing screen on this probe in such a way that the surgeon can view the zone on which he is operating without having to look away.

More precisely, a first subject of the invention is an optical probe for medical applications, devised so as to be able to be held in one hand, the said probe comprising at least:
a first so-called excitation lighting source suitable for causing a fluorescence radiation of predetermined substances,
a second visible lighting source, the first and the second source being devised so as to illuminate a common zone termed the intervention zone;
an optical objective;
a monoblock splitter prismatic assembly and spectral filters;
a first photosensitive matrix sensor;
a second photosensitive matrix sensor;
   the optical objective, the monoblock splitter prism, the spectral filters, the first and second photosensitive matrix sensors being devised in such a way that, when the optical objective is arranged a predetermined distance from the intervention zone, the image in the fluorescence spectrum of the said zone given by the objective is formed on the photosensitive surface of the first matrix sensor and the image in the visible spectrum of the said zone given by the objective is formed on the photosensitive surface of the second matrix sensor.

Advantageously, the splitter prismatic assembly is a splitter cube comprising a dichroic treatment reflecting the visible radiation and transmitting the radiation lying in the fluorescence band or vice versa, the first and second photosensitive matrix sensors being arranged on two perpendicular faces of the splitter cube.

Advantageously, the second visible lighting source comprises a polarizing filter, an analyser then being arranged between the monoblock splitter prism and the second photosensitive matrix sensor, the direction of polarization of the analyser then being perpendicular to the direction of polarization of the polarizing filter.

Advantageously, the first matrix sensor is associated with a first filter, transmitting solely in the fluorescence band, and that the second matrix sensor is associated with a second filter, transmitting visible wavelengths with the exception of those included in the fluorescence band.

A second subject of the invention is an optical probe for medical applications, devised so as to be able to be held in one hand, the said probe comprising at least:
a first so-called excitation lighting source suitable for causing a fluorescence radiation of predetermined substances,
a second visible lighting source, the first and the second source being devised so as to illuminate a common zone termed the intervention zone;
an optical objective;
a monoblock splitter prismatic assembly and spectral filters;
a photosensitive matrix sensor;
   the optical objective, the monoblock splitter prism, the spectral filters, the photosensitive matrix sensor being devised in such a way that, when the optical objective is arranged a predetermined distance from the intervention zone, the image in the fluorescence spectrum of the said zone given by the objective is formed on the first part of the photosensitive surface of the matrix sensor and the image in the visible spectrum of the said zone given by the objective is formed on a second part of the photosensitive surface of the matrix sensor.

Advantageously, the splitter prismatic assembly comprises a splitter cube and a deflecting prism and a compensation plate, the splitter cube comprising a dichroic treatment reflecting the visible radiation and transmitting the radiation lying in the fluorescence band or vice versa.

Advantageously, the splitter prismatic assembly is a “Koster” prism composed of two identical bracket prisms, the face common to the two prisms comprising a dichroic treatment reflecting the visible radiation and transmitting the radiation lying in the fluorescence band or vice versa.

Advantageously, the second visible lighting source is associated with a polarizing filter, an analyser then being arranged between the splitter prismatic assembly and the second half of the photosensitive matrix sensor, the direction of polarization of the analyser then being perpendicular to the direction of polarization of the polarizing filter.

A third subject of the invention is an optical probe for medical applications, devised so as to be able to be held in one hand, the said probe comprising at least:
a first so-called excitation lighting source suitable for causing a fluorescence radiation of predetermined substances,
a second visible lighting source, the first and the second source being devised so as to illuminate a common zone termed the intervention zone;
a first photosensitive matrix sensor;
a second photosensitive matrix sensor;

the first and second photosensitive matrix sensors being devised in such a way that, when the probe is arranged a predetermined distance from the intervention zone, the image in the fluorescence spectrum of the said zone is formed on the photosensitive surface of the first matrix sensor and the image in the visible spectrum of the said zone is formed on the photosensitive surface of the second matrix sensor, characterized in that the second visible lighting source comprises a polarizing filter, an analyser then being arranged upstream of the second photosensitive matrix sensor, the direction of polarization of the analyser then being perpendicular to the direction of polarization of the polarizing filter.

Advantageously, the first matrix sensor is associated with a first filter, transmitting solely in the fluorescence band, and the second matrix sensor is associated with a second filter, transmitting visible wavelengths with the exception of those included in the fluorescence band.

Advantageously, the first so-called excitation lighting source is a laser source whose spectral emission corresponds to the excitation spectrum of the fluorophore.

Advantageously, the probe comprises means for measuring the inclination of the optical probe and means for cutting off the laser source when the inclination of the optical probe exceeds a predetermined value.

Advantageously, the second lighting source is at least one so-called “white” light-emitting diode comprising a filter cutting off the fluorescence spectrum.

Advantageously, the second lighting source comprises a plurality of filtered white diodes arranged in a regular manner around the optical objective.

Finally, the probe can comprise an imager devised so as to display either the image in the visible spectrum of the intervention zone, or the image in the fluorescence spectrum of the intervention zone, or a superposition of these two images, the said images emanating from the photosensitive matrix sensor or sensors.

### BRIEF DESCRIPTION OF THE DRAWINGS

The invention will be better understood and other advantages will become apparent on reading the nonlimiting description which follows and by virtue of the appended figures among which:

FIG. 1 represents a first embodiment of a probe according to the invention and of the ancillary devices;

FIG. 2 represents a second embodiment of a probe according to the invention, only the upper part of the probe is represented in this figure;

FIG. 3 represents a variant of this second embodiment of a probe according to the invention;

FIG. 4 represents the general principle of the device making it possible to ensure ocular safety of the probe;

FIG. 5 represents a probe according to the invention comprising a display;

FIG. 6 represents an embodiment of the visible lighting;

FIG. 7 represents the spectral distribution of the various necessary optical filterings in a probe according to the invention.

### DETAILED DESCRIPTION

As stated, the probes according to the invention comprise two pathways, a first pathway dedicated to fluorescence radiation and a second pathway dedicated to visible radiation. There exist various possible optical architectures making it possible to produce these two pathways. A first possible architecture consists in producing two distinct pathways each comprising their own source, their optical system and their matrix sensor, each pathway forming an image of the same intervention zone. The visible pathway then operates under polarized light.

A second possible optical architecture making it possible to ensure greater compactness of the probe is to produce an optical combination comprising a single optical objective which is common to the two pathways.

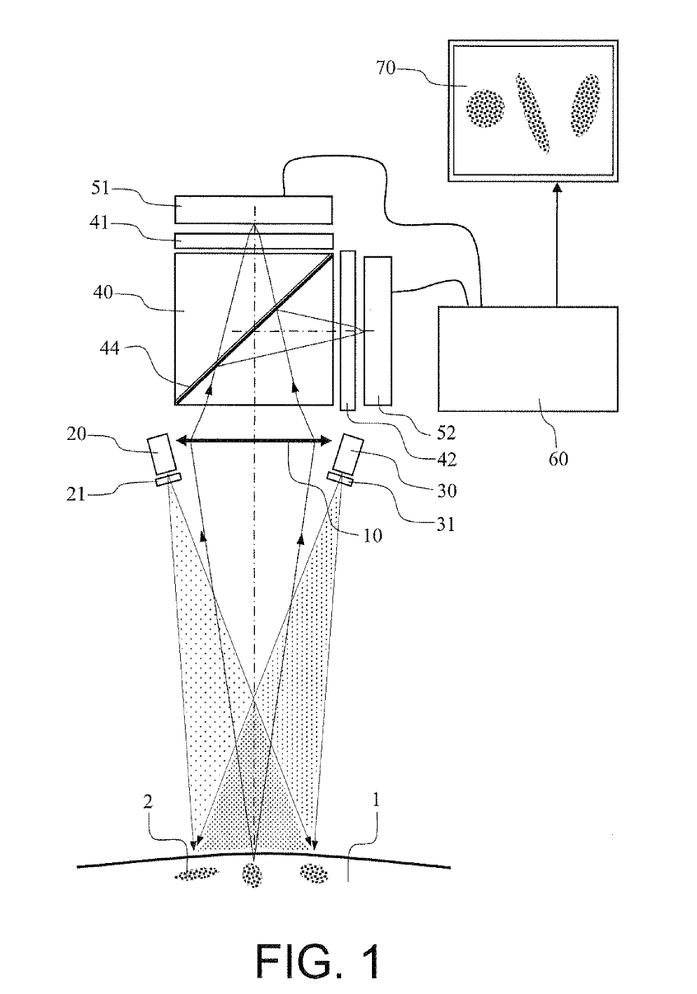

FIG. 1 represents the diagram of a first embodiment of a peroperative optical probe according to this architecture. In this and in the following figures, the dimensions are not necessarily representative of those of a real probe, the objective being to show the general opto-mechanical principles of implantation of the various elements and the route of the light rays through the optical elements. In this and the following figures, neither the mechanical casing surrounding the probe and held by the user nor the various mechanical supports making it possible to maintain and to position the various optical components are represented. The placing of its various elements does not pose any particular difficulties for the person skilled in the art.

The probe is represented in operational use, that is to say held by an operator above an intervention zone.

The probe according to the invention essentially comprises:
a first so-called excitation lighting source 20, the spectral radiation of this first source is filtered by means of the optical filter 21;
a second visible or “white” lighting source 30 filtered by means of the optical filter 31, the first and the second source being devised so as to illuminate a common zone termed the intervention zone 1. In FIG. 1, the various radiations emitted are represented dotted;
an optical objective 10 represented conventionally by a double arrow;
a monoblock splitter prismatic assembly 40 and spectral filters 41 and 42;
a first photosensitive matrix sensor 51;
a second photosensitive matrix sensor 52;

The first and the second source constitute the emission pathway of the probe. The objective, the spectral filters and the matrix sensors make up the two reception pathways of the optical probe.

The intervention zone 1 may be, for example, a part of an abdominal cavity that may comprise cancerous tissues 2. It has been previously treated by means of an injection of an antibody-fluorophore conjugate which has fixed itself to the diseased cells 2. The excitation source 20 emits a spectral radiation in a first so-called excitation spectral band which illuminates the intervention zone 1, making it possible to obtain the fluorescence of the diseased cells marked by the fluorophore. The fluorescence radiation is emitted in a second so-called fluorescence spectral band. Generally, the gist of the fluorescence spectrum is emitted in the red and the near-infrared.

The intervention zone is also illuminated by so-called “white” visible light coming from the source 30. This light is filtered and no longer comprises radiation lying in the red or the near infrared. Thus, it is possible to spectrally split the visible spectrum from the fluorescence spectrum. Hence, the probe comprises two reception pathways, the first dedicated to visible radiation, the second to fluorescence radiation. These two pathways comprise a common optical objective 10 which forms on the one hand, the fluorescence image of the intervention zone 1 on the first photosensitive matrix sensor 51 and the visible image of the intervention zone on the second photosensitive matrix sensor 52, the spectral splitting of the two images is ensured by a dichroic treatment 44 arranged inside the monoblock splitter prism 40. It is possible to invert the reception pathways to obtain better implantation. Additional spectral filters 41 and 42 allow perfect splitting of the spectra. The electronic images provided by these two sensors are processed by an electronic processing unit 60, independent of the probe which can ensure the usual processings of images and which returns the processed images to a viewing screen 70 which can either display one of the two images, visible or fluorescence, or can display a fusion of these two images. The fusion of these two images may be the image in the visible spectrum, on which the most intense parts of the fluorescence image are superimposed, these parts possibly being for example colour tinted.

A simple dichroic semi-reflecting plate can, of course, be used as prismatic assembly to spectrally split the two images. However, it is preferable to use a splitter cube 40 as seen in FIG. 1 whose internal face comprises a dichroic treatment 44. Indeed, the splitter cube exhibits numerous advantages. On the one hand, it behaves as a thick glass plate and makes it possible to reduce the proportions of the optical beams, the refraction inside the cube giving rise to less divergence of the beams. Thereafter, it makes it possible to increase the back-focus of the objective, making it possible to use standard optics. It is demonstrated that if e is the thickness of the cube and n its refractive index, the increase in back-focus δT equals:

$$e \cdot ( 1 - \frac{1}{n} ) .$$

Finally, it is possible to cement the filters and the sensors onto the plane faces of the cube. On the one hand, this eliminates possible nuisance images and on the other hand produces a component which will be very insensitive to mechanical knocks or to thermal variations, this being essential for a probe which is handled constantly. Cementing the filters and sensors, or indeed the objective, onto the cube makes it possible to increase the robustness of the probe, and to prevent any inadvertent maladjustments.

In the basic version illustrated in FIG. 1, the sensors 51 and 52 are different. It is thus possible to specifically adapt their spectral response to the radiation received. In a variant, it is possible to use a single sensor 53 to produce the visible and fluorescence images. The image in the fluorescence spectrum of the said zone is formed on the first part 54, for example a first half of the photosensitive surface of the matrix sensor 53, and the image in the visible spectrum of the said intervention zone is formed on a second part 55, for example a second half, of the photosensitive surface of the matrix sensor. In this case, the splitter prism must have a particular arrangement so as to ensure the focusing of the two images of the intervention zone in the plane of the photosensitive surface of the matrix sensor.

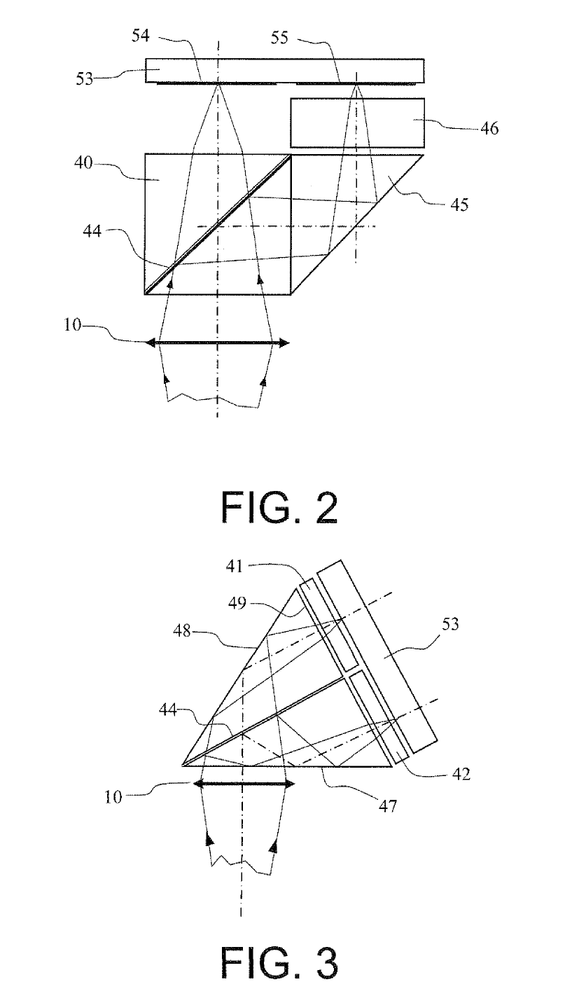

In a first embodiment represented in FIG. 2, the splitter prism comprises a splitter cube 40, a deflecting prism 45 and a compensation plate 46, this plate also being able to act as filter of the fluorescent light, or be cemented to such a filter. The splitter cube 40 comprises as previously a dichroic internal face 44. The deflecting prism 45 makes it possible to orient the optical axis on the reflection pathway of the dichroic face along an axis parallel to that of the transmission pathway. It naturally operates by total internal reflection. The compensator plate 46 makes it possible to equalize the back-focuses on the two reception pathways. Indeed, it is necessary to compensate the optical path lost in the deflecting prism 45 through an increase in the back-focus due to the compensator plate 46. Although not represented in FIG. 2, this embodiment can comprise a filter 41, adapted to the fluorescence spectral band, as well as a filter 42, whose spectral band comprises visible wavelengths with the exception of those contained in the fluorescence band.

In a second embodiment represented in FIG. 3, the splitter prism is a “Koster” prism composed of two identical prisms, the face common to the two prisms 44 comprising a dichroic treatment reflecting the visible radiation and transmitting the radiation lying in the near infrared. Each prism has a cross-section of right-angled triangular shape. The propagation of the light rays is as follows: the light rays emanating from the objective 10 enter the “Koster” prism through the lower face 47, are split by the dichroic treatment 44, are reflected by total internal reflection on the faces 47 and 48 and exit at quasi-normal incidence through the face 49 where they may be filtered by the filters 41 and 42 before being focused on the single photosensitive sensor 53.

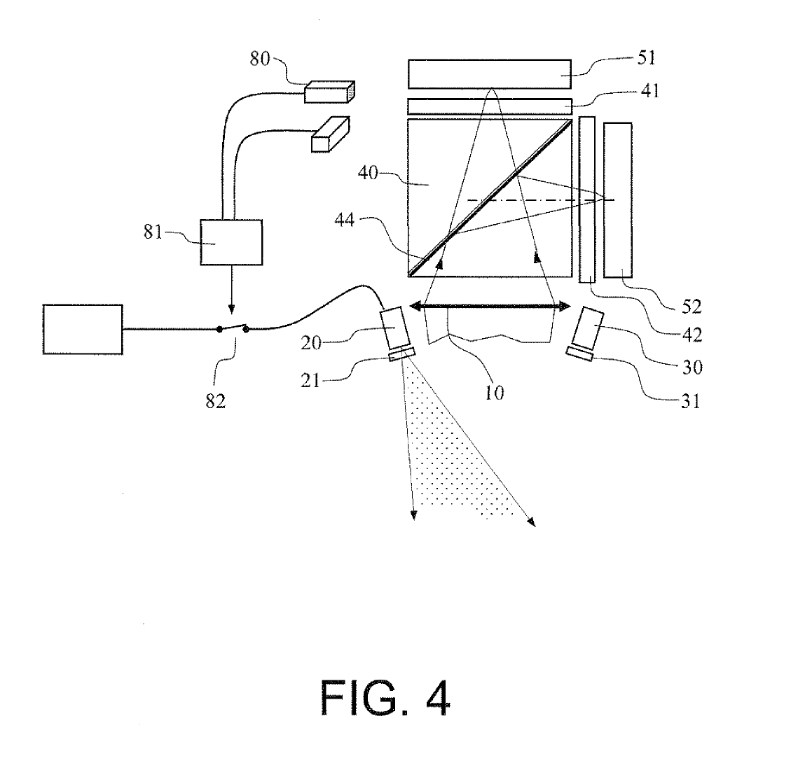

As was stated, the excitation sources are generally laser sources powerful enough to cause discernable fluorescence. Hence, it is important to ensure the ocular safety of the operator or of the personnel undertaking the intervention. A simple solution is set forth in FIG. 4. Two inclinometers 80 or equivalent devices oriented at 90 degrees to one another are fixed to the probe. These inclinometers are linked to a processing device 81 which compares the probe's inclination measurements with predetermined so-called safety inclination values. When the measurements attain or exceed the said values, the processing device 81 cuts off the laser beam. The safety value can correspond, for example, to a horizontal placement of the probe. This cutting off is represented symbolically by the breaker 82. Either the laser's power supply can be cut off electrically, or a cover can be introduced in front of the beam if it is not desired to abruptly interrupt the laser emission.

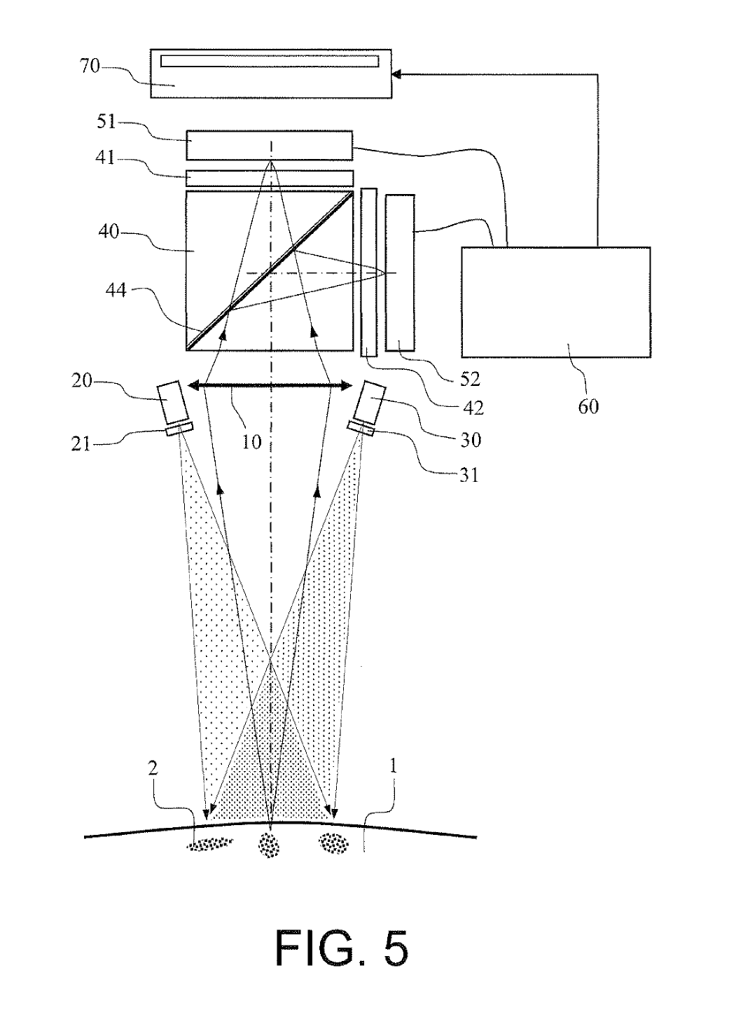

The images provided by the sensor or sensors are displayed on a viewing screen 70. This screen may be a screen fixed permanently in the operating theatre. It is also possible to fix a screen of small size directly on the optical probe in such a way that an image of the intervention zone is constantly under the operator's eyes, this being illustrated in FIG. 5. This screen 70 may be, for example, a liquid-crystal flat screen. This screen may also be filtered so as not to disturb the fluorescence.

By way of nonlimiting example, the optical, photometric, geometric characteristics of the various optical and opt-electronic components may be as follows.

Characteristics of the Excitation Source

The excitation source 20 is adapted to the excitation spectrum of the fluorophore used. The expression excitation spectrum is of course understood to mean the fluorescence excitation spectrum of the fluorophore. In the case where the dye is a derivative of indocyanine green, known by the acronym ICG having an optimal absorption at 686 nm and an emission at 704 nm, the source may be a laser selected to emit at 685 nm±5 nm. This laser is temperature-regulated by the Peltier effect so as to stabilize the emission wavelength at the desired wavelength. By way of example, a one degree variation in the temperature makes it possible to displace the emission peak by 0.2 nm. FIG. 7 represents a certain number of spectral distributions as a function of wavelength, the latter lying between 400 and 900 nm. The curve in grey E_{L} centred on 685 nm represents the spectral emission of the laser. An interferential filter can be used to improve the spectral purity of the laser emission. This makes it possible to avoid the presence of secondary emissions of the excitation source in the fluorescence spectral band. The spectral transmission curve R_{L} of this filter is represented in FIG. 7. It is centred on 685 nm and has a mid-height width of 30 nm. By nature, the filtering curve of this type of interferential filter is very sensitive to the angle of incidence of the light rays which pass through the filter. Hence, it is preferable to use them under collimated light. It suffices to place a collimating lens, for example a gradient-index lens, in front of the emission source and to arrange the interferential filter behind this lens. It is thereafter possible to arrange either another lens, or a diffuser to obtain the desired divergence making it possible to illuminate the intervention zone homogeneously.

So as to avoid increasing the weight and the volume of the probe which must be held easily by the user, it is preferable to site the laser away from the probe and to convey the excitation radiation by means of an optical fibre. To obtain easily detectable fluorescence, the excitation power must lie between 0.25 W and 0.5 W. In this case, it is recommended to use an ocular safety device with clinometers, such as was described previously.

Characteristics of the Visible Source or White Source.

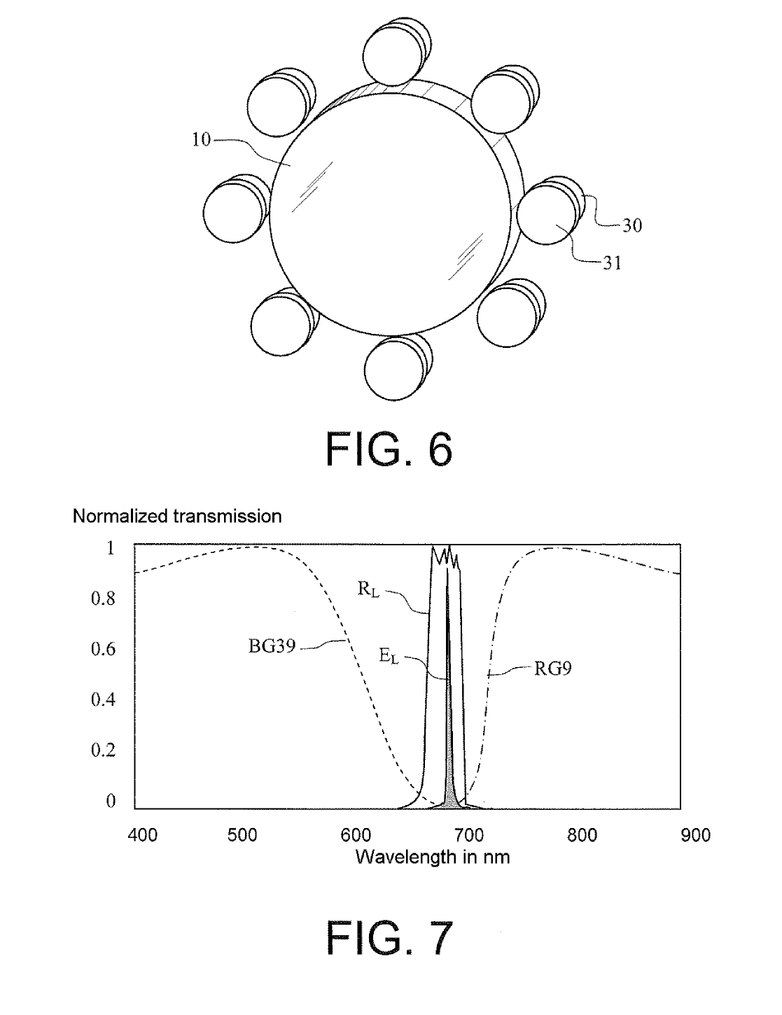

It is preferable to use filtered white light-emitting diodes to produce this lighting. White diodes make it possible to obtain high luminous powers within reduced proportions. So as to obtain a very homogeneous lighting distribution, it is possible to use several white sources 30 distributed uniformly around the input optic of the objective 10 as represented in FIG. 6 where, by way of example, 8 filtered light-emitting diodes surround the optical objective. It is very desirable that these diodes are filtered so as to eliminate the spectral band of their emission spectrum corresponding to the emission spectrum of the fluorophore, or fluorescence spectrum (red and infrared in the present case) which would disturb the generally very weak fluorescence radiation. For this purpose it is possible to use filters 31 of “BG39” type from the company SCHOTT which operate by absorption and are therefore insensitive to the inclination of the light rays and which exhibit the advantage of preserving good colour rendition. When the device comprises a ring of diodes, it may be beneficial to arrange a single filter as an annulus or as a portion of an annulus in front of the assembly of lighting diodes. The spectral transmission curve BG39 of this filter is represented in FIG. 7.

Having regard to the low intensity of the fluorescence radiation, it is preferable to use just the filtered lighting source to illuminate the intervention zone and to totally cut off the scialytic lighting of the room where the intervention is taking place, the latter being anyway weakly illuminated by the display screen which may also be filtered if it is of reduced dimensions.

Biological tissues are moist media and may, moreover, be moistened during the intervention by serum. Experimental trials have shown that when they are illuminated by white light sources such as described hereinabove, they can behave as a reflecting surface, and give rise to significant specular reflection, liable to appear on the image acquired in the visible spectrum, then causing an impediment for the user. Indeed, the spots of specular reflection appearing on the visible image are blurred, since the virtual object to which they correspond is approximately twice as far away as the focusing distance. It has been noted that these spots may be very intense with respect to the white light having diffused, producing local saturations on the imager. This problem is remedied by incorporating a filter polarizing in proximity to the white light source, and by placing an analyser in front of the photosensitive matrix corresponding to the visible image, the direction of polarization of this analyser being perpendicular to that of the polarizing filter placed in proximity to the white lighting source. This refinement makes it possible to eliminate, upstream of the imager, the signal due to specular reflections since the specular reflected light retains its polarization, unlike the diffuse reflected light, the latter being depolarized.

It is for example possible to use a first linear polarization filter with the “Vikuiti” brand and of HN type 32, and an analyser consisting of a filter of identical design, oriented perpendicularly to the first filter. In order not to influence the fluorescence signal, the analyser is arranged after the splitting of the visible and fluorescence signals. Preferably, the analyser is arranged against the visible imager 52 or against the filter 42 associated with this imager. A polarizing filter and an analyser of small thickness, typically a few hundred μm, are preferably chosen.

Opto-Mechanical and Photometric Characteristics

The intervention zone has a diameter of about 80 mm. The working distance of a hand-held optical probe, that is to say the distance which separates the objective from the intervention zone is of the order of 120 to 150 mm. To maintain significant compactness, photosensitive matrix sensors whose diagonal is close to 8 mm are chosen. The focal length of the objective 10, which must be about 12.5 mm, is deduced from these dimensions. The objective adopted may be derived from the standard objectives used for photographic snapshots. The objective must be designed in such a way that it is possible to interpose the splitter prismatic assembly between the last dioptre of the objective and the surface or surfaces of the photosensitive sensors. This constraint does not pose any particular problems in so far as the splitter assembly, that may be regarded as a thick glass plate, naturally introduces an increase in the back-focus of the objective.

As was stated, the spectral filtering of the two reception pathways must be treated carefully so as to split the fluorescence spectrum perfectly from the visible spectrum. The dichroic treatment of the splitter prism does not necessarily ensure this splitting perfectly. Hence, it may be judicious to arrange in front of each photosensitive matrix sensor an optical filter transmitting solely either the visible, but with the exception of the spectral band of the fluorescent emission (or fluorescence band), corresponding to the fluorescence spectrum, or solely in the spectral band of the fluorescence emission. In numerous applications, this fluorescence band is situated in the red or in the near infra-red, but the invention may be readily adapted to other fluorescence spectra. In the first case, it is possible to use a filter of “BG39” type, in the second case, it is possible to use an RG9 filter, also from the company SCHOTT. This company's technical sheets may be referred to in order to obtain all the optical characteristics of these filters. By way of information, the spectral transmission curve RG9 of this filter is represented in FIG. 7. So as to improve the compactness, the quality and the robustness of the optical assembly, it is advantageous to cement the spectral selection filters onto the faces of the splitter prismatic assembly opposite the photosensitive surfaces of the sensors.

Characteristics of the Matrix Sensors

The sensors may be of CCD type, the acronym standing for “Charge Coupled Device” or CMOS type, the acronym standing for “Complementary Metal Oxide Semiconductor”.

When the probe comprises two different sensors, the matrix sensor arranged on the fluorescence pathway may be monochrome. It must have a good resolution and good sensitivity in the spectrum lying in the red and the near infrared. By way of example, the matrix sensor referenced “ICX285AL” from the company SONY meets this requirement. It possesses a useful surface of length 8.3 mm and of width 6.6 mm comprising 1292 rows of 1024 pixels, each square pixel measuring 6.45 μm by 6.45 μm. It offers a good quantum yield at 700 nm. This sensor does not need any cooling and can operate at rates of several images per second. The manufacturer's sheet may be referred to for all further information.

The matrix sensor arranged on the visible pathway is necessarily a colour sensor and its dimensional characteristics must be much like those of the matrix sensor arranged on the fluorescence pathway. By way of example, a matrix sensor of CMOS type from the Canadian company PIXELLINK meets this requirement. It possesses a useful surface of length 8.6 mm and of width 6.8 mm comprising 1280 rows of 1024 pixels, each square pixel measuring 6.7 μm by 6.7 μm. It operates at a rate of 24 images per second.

When the probe comprises a single sensor, the latter must necessarily be a colour sensor whose photosensitive surface is of rectangular shape. By way of example, the matrix sensor referenced “ICX412AQ” from the company SONY meets this requirement. The manufacturer's sheet may be referred to for all further information.

So as to improve the compactness, the quality and the robustness of the optical assembly, it is also advantageous to cement the sensors onto the spectral selection filters. It is of course very important that the visible and fluorescence images be perfectly superimposed. The alignment and the adjustment for superimposing the two photosensitive surfaces can be done by means of a stereomicroscope with high precision and do not present any particular difficulties. Here again, it is preferable to cement the sensors onto the optical elements so as to preserve the superimposed positions. It is also preferable that all or part of the control electronics for the sensors be sited elsewhere so as to lighten the probe and to reduce its proportions.

Ultimately, it is possible to produce a portable optical probe whose length does not exceed 170 mm and whose rectangular cross-section has dimensions of 56×43 mm and whose weight does not exceed 500 g.

1. An optical probe for medical applications, devised so as to be able to be held in one hand, comprising:
a first excitation lighting source suitable for causing a fluorescence radiation of predetermined substances,
a second visible lighting source, the first and the second source being devised so as to illuminate a common zone termed the intervention zone;
an optical objective;
a monoblock splitter prismatic assembly and spectral filters;
a first photosensitive matrix sensor;
a second photosensitive matrix sensor;
the optical objective, the monoblock splitter prism, the spectral filters, the first and second photosensitive matrix sensors being devised such that, when the optical objective is arranged a predetermined distance from the intervention zone, the image in a fluorescence spectrum of the said zone given by the objective is formed on a photosensitive surface of the first matrix sensor and the image in the visible spectrum of the said zone given by the objective is formed on a photosensitive surface of the second matrix sensor.

2. The optical probe according to claim 1, wherein the splitter prismatic assembly is a splitter cube comprising a dichroic treatment reflecting the visible radiation and transmitting the radiation lying in the fluorescence band or vice versa, the first and second photosensitive matrix sensors being arranged on two perpendicular faces of the splitter cube.

3. The optical probe according to claim 1, wherein the second visible lighting source further comprises a polarizing filter, an analyser then being arranged between the monoblock splitter prism and the second photosensitive matrix sensor, the direction of polarization of the analyser then being perpendicular to the direction of polarization of the polarizing filter.

4. The optical probe according to claim 1, such that the first matrix sensor is associated with a first filter, transmitting solely in the fluorescence band, and that the second matrix sensor is associated with a second filter, transmitting visible wavelengths with the exception of those included in the fluorescence band.

5. The optical probe according to claim 1, wherein the first excitation lighting source is a laser source whose spectral emission corresponds to the excitation spectrum of the fluorophore.

6. The optical probe according to claim 5, wherein the probe further comprises means for measuring the inclination of the optical probe and means for cutting off the laser source when the inclination of the optical probe exceeds a predetermined value.

7. The optical probe according to claim 1, wherein the second lighting source is at least one white light-emitting diode comprising a filter cutting off the fluorescence spectrum.

8. The optical probe according to claim 7, wherein the second lighting source further comprises a plurality of filtered white diodes arranged in a regular manner around the optical objective.

9. The optical probe according to claim 1, wherein the probe further comprises an imager devised so as to display either the image in the visible spectrum of the intervention zone, or the image in the fluorescence spectrum of the intervention zone, or a superposition of these two images, the said images emanating from the photosensitive matrix sensor or sensors.

10. An optical probe for medical applications, devised so as to be able to be held in one hand, comprising:
a first excitation lighting source suitable for causing a fluorescence radiation of predetermined substances,
a second visible lighting source, the first and the second source being devised so as to illuminate a common zone termed the intervention zone;
an optical objective;
a monoblock splitter prismatic assembly and spectral filters;
a photosensitive matrix sensor;
the optical objective, the monoblock splitter prism, the spectral filters, the photosensitive matrix sensor being devised in such a way that, when the optical objective is arranged a predetermined distance from the intervention zone, the image in the fluorescence spectrum of the said zone given by the objective is formed on a first part of the photosensitive surface of the matrix sensor and the image in the visible spectrum of the said zone given by the objective is formed on a second part of the photosensitive surface of the matrix sensor.

11. The optical probe according to claim 10, wherein the splitter prismatic assembly further comprises a splitter cube and a deflecting prism and a compensation plate, the splitter cube comprising a dichroic treatment reflecting the visible radiation and transmitting the radiation lying in the fluorescence band or vice versa.

12. The optical probe according to claim 10, wherein the splitter prismatic assembly is a “Koster” prism composed of two identical bracket prisms, the face common to the two prisms comprising a dichroic treatment reflecting the visible radiation and transmitting the radiation lying in the fluorescence band or vice versa.

13. The optical probe according to claim 10, wherein the second visible lighting source is associated with a polarizing filter, an analyser then being arranged between the splitter prismatic assembly and the second half of the photosensitive matrix sensor, the direction of polarization of the analyser then being perpendicular to the direction of polarization of the polarizing filter.

14. The optical probe according to claim 10, wherein the first excitation lighting source is a laser source whose spectral emission corresponds to the excitation spectrum of the fluorophore.

15. The optical probe according to claim 14, wherein the probe further comprises means for measuring the inclination of the optical probe and means for cutting off the laser source when the inclination of the optical probe exceeds a predetermined value.

16. The optical probe according to claim 10, wherein the second lighting source is at least one white light-emitting diode comprising a filter cutting off the fluorescence spectrum.

17. The optical probe according to claim 16, wherein the second lighting source further comprises a plurality of filtered white diodes arranged in a regular manner around an optical objective.

18. The optical probe according to claim 10, wherein the probe further comprises an imager devised so as to display either the image in the visible spectrum of the intervention zone, or the image in the fluorescence spectrum of the intervention zone, or a superposition of these two images, the said images emanating from the photosensitive matrix sensor or sensors.

19. An optical probe for medical applications, devised so as to be able to be held in one hand, comprising:
a first excitation lighting source suitable for causing a fluorescence radiation of predetermined substances,
a second visible lighting source, the first and the second source being devised so as to illuminate a common zone termed the intervention zone;
a first photosensitive matrix sensor;
a second photosensitive matrix sensor;
the first and second photosensitive matrix sensors being devised in such a way that, when the probe is arranged a predetermined distance from the intervention zone, the image in the fluorescence spectrum of the said zone is formed on a photosensitive surface of the first matrix sensor and the image in the visible spectrum of the said zone is formed on a photosensitive surface of the second matrix sensor,
wherein the second visible lighting source comprises a polarizing filter, an analyser then being arranged upstream of the second photosensitive matrix sensor, the direction of polarization of the analyser then being perpendicular to the direction of polarization of the polarizing filter.

20. The optical probe according to claim 19, such that the first matrix sensor is associated with a first filter, transmitting solely in the fluorescence band, and that the second matrix sensor is associated with a second filter, transmitting visible wavelengths with the exception of those included in the fluorescence band.

21. The optical probe according to claim 19, wherein the first excitation lighting source is a laser source whose spectral emission corresponds to the excitation spectrum of the fluorophore.

22. The optical probe according to claim 19, wherein the probe further comprises means for measuring the inclination of the optical probe and means for cutting off the laser source when the inclination of the optical probe exceeds a predetermined value.

23. The optical probe according to claim 19, wherein the second lighting source is at least one white light-emitting diode comprising a filter cutting off the fluorescence spectrum.

24. The optical probe according to claim 23, wherein the second lighting source further comprises a plurality of filtered white diodes arranged in a regular manner around the optical objective.

25. The optical probe according to claim 19, wherein the probe further comprises an imager devised so as to display either the image in the visible spectrum of the intervention zone, or the image in the fluorescence spectrum of the intervention zone, or a superposition of these two images, the said images emanating from the photosensitive matrix sensor or sensors.
